(19) United States
(12) Patent Application Publication
(10) Pub. No.: US 2016/0166388 A1
(43) Pub. Date: Jun. 16, 2016

(54) COMPOSITE METALLIC-CERAMIC IMPLANTS AND RELATED METHODS

(71) Applicant: Amedica Corporation, Salt Lake City, UT (US)

(72) Inventors: Bhajanjit Singh Bal, Columbia, MO (US); Bryan J. McEntire, Sandy, UT (US); Ramaswamy Lakshminarayanan, West Jordan, UT (US)

(21) Appl. No.: 14/570,916

(22) Filed: Dec. 15, 2014

(51) Int. Cl. A61F 2/30; A61F 2/38; A61F 2/36

(52) U.S. Cl. CPC A61F 2/30734; A61F 2/3609; A61F 2/3859; A61F 2/30942; A61F 2002/365; A61F 2002/3617

(57) ABSTRACT

Embodiments of apparatus, systems, and methods relating to biomedical implants and other devices made up of a composite of materials comprising metal and/or metal alloys and ceramics. In some embodiments, a modular biomedical implant may comprise a first metallic member comprising at least one of a metal and a metal alloy, a second metallic member comprising at least one of a metal and a metal alloy, and a monolithic ceramic insert comprising a ceramic material positioned in between the first metallic member and the second metallic member so as to at least substantially prevent contact between the first metallic member and the second metallic member.

### SUMMARY

Disclosed herein are embodiments of apparatus, methods, and systems relating to biomedical implants comprising a composite of materials comprising metal and/or metal alloys and ceramics. In some embodiments, the biomedical implants may be modular, and may be configured such that a ceramic material is positioned in between two metallic materials of the modular implant to prevent certain negative outcomes associated with directly interfacing metallic pieces, such as pitting, crevice, fretting, and galvanic corrosion, which may lead to taper corrosion. Various embodiments and implementations are contemplated for utilization in any biomedical implant system wherein two similar or dissimilar modular metal components need to be mechanically attached or otherwise placed in contact with one another.

In a more particular example of a modular biomedical implant according to one embodiment, the implant may comprise a first metallic member comprising at least one of a metal and a metal alloy, a second metallic member comprising at least one of a metal and a metal alloy, and a ceramic insert (in some embodiments, a monolithic ceramic insert), such as a sleeve, comprising a ceramic material. The insert may be positioned in between the first metallic member and the second metallic member so as to at least substantially prevent contact between the first metallic member and the second metallic member. In some embodiments, the insert may be positioned in between the first metallic member and the second metallic member so as to fully prevent contact between the first metallic member and the second metallic member.

In some embodiments, the ceramic material may comprise, for example, at least one of alumina, zirconia, zirconia-toughened alumina, silicon nitride, and silicon carbide. One or both of the metallic members may comprise, for example, cobalt-chromium, titanium-aluminum-vanadium, and/or zirconium-niobium alloys

The first metallic member may comprise, for example, a femoral head of a modular hip implant. Similarly, the second metallic member may comprise a neck of the modular hip implant, such as a neck of an elongated stem configured for being coupled with a patient's femur, the neck of which is configured to be coupled with the femoral head. In some such embodiments, the monolithic ceramic insert may comprise a sleeve positioned within a cavity formed within the femoral head. The cavity may comprise a reverse taper such that a peripheral opening of the cavity comprises a first diameter and an internal portion of the cavity comprises a second diameter, wherein the first diameter is less than the second diameter.

In some such embodiments, the sleeve may also comprise a taper, such as an external reverse taper, that may be configured such that a first end of the sleeve has an external diameter of a first length and a second end of the sleeve opposite from the first end has an external diameter of a second length, wherein the first length is greater than the second length. The first end may be configured to be received within the cavity adjacent to the internal portion of the cavity. In some embodiments, the reverse taper of the sleeve may be configured to at least substantially match the reverse taper of the cavity.

In some embodiments, the monolithic ceramic insert may comprise a wall having a thickness of at least about 1 mm. For example, the wall thickness may be between about 1 mm and about 10 mm in some embodiments. This may be preferred in order to provide sufficient separation between the metallic components to prevent corrosion and other negative outcomes referenced herein. In some embodiments, the monolithic ceramic insert may comprise a sleeve, which may have an annular shape. In such embodiments, the annular wall of the sleeve may have a thickness of at least about 1 mm.

In some embodiments, the biomedical implant may comprise another type of implant, such as a knee implant. In some such embodiments, the first metallic member may comprise an articulating femoral component of a knee implant, and the second metallic member may comprise an intramedullary rod of the knee implant configured to be coupled with the femoral component.

Some embodiments may further comprise a third metallic member comprising at least one of a metal and a metal alloy; a fourth metallic member comprising at least one of a metal and a metal alloy; and a second monolithic ceramic insert comprising a ceramic material positioned in between the third metallic member and the fourth metallic member so as to at least substantially prevent contact between the third metallic member and the fourth metallic member. In some such embodiments, the first metallic member may comprise an articulating femoral component of a knee implant, and the second metallic member may comprise an intramedullary rod of the knee implant configured to be coupled with the femoral component. Similarly, in some such embodiments, the third metallic member may comprise a tibial component of the knee implant, and the fourth metallic member may comprise at least one of a rod, such as an intramedullary rod, and a metaphyseal cone of the knee implant.

In another specific example of a modular hip implant according to another embodiment, the implant may comprise a femoral head comprising at least one of a metal and a metal alloy. The femoral head may further comprise a cavity, and the cavity may comprise a peripheral opening having a first diameter and a terminal portion having a second diameter greater than the first diameter. The implant may further comprise a ceramic insert comprising a ceramic material (in some embodiments, a monolithic ceramic insert) positioned within the cavity so as to at least substantially prevent contact between a portion of the femoral head defining the cavity and a second metallic member of the modular hip implant, such as a neck of an elongated stem of the implant.

The ceramic insert may comprise an exterior surface configured to engage the portion of the femoral head defining the cavity and an interior surface configured to engage the second metallic member, wherein the exterior surface extends between a first end and a second end, and wherein a diameter of the exterior surface at the first end is greater than a diameter of the exterior surface at the second end.

In some embodiments, the cavity may define a reverse taper extending from the peripheral opening to the terminal portion. Similarly, the exterior surface of the ceramic insert may define a reverse taper, which reverse taper may match, or at least substantially match, the reverse taper of the cavity.

Thus, in some embodiments, the reverse taper of the cavity may extend at a first angle relative to a central axis of the cavity, and the reverse taper of the ceramic insert may extend at a second angle relative to a central axis of the ceramic insert. The the first angle may be at least substantially identical to the second angle such that the reverse tapers match.

In embodiments comprising a hip stem comprising a neck, the hip stem may be configured to be coupled with an upper end of a patient's femur, and the neck may be configured to be received in the ceramic insert.

In some embodiments, the ceramic insert may comprise a sleeve. In some such embodiments, the sleeve may define an annular shape. In some embodiments, the sleeve may comprise a wall having a thickness greater than about 1 mm.

In a particular example of a method for manufacturing a biomedical implant according to one implementation, the method may comprise providing a first metallic member comprising at least one of a metal and a metal alloy, wherein the first metallic member comprises a cavity comprising a peripheral opening and an internal portion, and wherein the cavity comprises a reverse taper such that the peripheral opening comprises a first diameter and the internal portion comprises a second diameter greater than the first diameter. A second metallic member comprising at least one of a metal and a metal alloy configured to be coupled with the first metallic member may also be provided, along with a ceramic insert comprising a ceramic material. In some implementations, the ceramic insert may comprise a monolithic ceramic insert, such as a silicon nitride ceramic insert.

In some implementations, the ceramic insert may comprise an external reverse taper such that a first end of the ceramic insert comprises an external diameter of a first length and a second end of the ceramic insert opposite from the first end has an external diameter of a second length, wherein the first length is greater than the second length, and wherein the first end is configured to be received within the cavity adjacent to the internal portion of the cavity.

The ceramic insert may be positioned in the cavity such that the ceramic insert at least partially lines the cavity. In some implementations, the ceramic insert may be positioned to fully line the cavity such that contact is prevented between the first metallic member and the second metallic member. In some implementations, the ceramic insert may be bonded to the first metallic member within the cavity. This may be accomplished, for example, by shrink-fitting, press-fitting, diffusion bonding, or cementing the ceramic insert to the first metallic member.

The second metallic member may then be inserted into the cavity defined at least in part by the ceramic insert so as to prevent, or at least limit, contact between the two metallic components.

In implementations comprising shrink-fitting the ceramic insert to the first metallic member, some such implementations may comprise heating the first metallic member to expand the dimensions of the cavity and create an expanded cavity; positioning the ceramic insert within the expanded cavity; and allowing the first metallic member to cool with the ceramic insert within the expanded cavity such that the first metallic member shrinks around the ceramic insert.

The features, structures, steps, or characteristics disclosed herein in connection with one embodiment or implementation may be combined in any suitable manner in one or more alternative embodiments or implementations.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 is an exploded view of a metallic femoral head and a ceramic insert of a modular hip implant according to one embodiment.

FIG. 2 depicts the metallic femoral head and a ceramic insert of the modular hip implant of FIG. 1 after the insert has been engaged with the femoral head.

FIG. 3 depicts the metallic femoral head and a ceramic insert of the modular hip implant of FIG. 1 along with a metallic, elongated stem of the modular hip implant

FIG. 4 is a flow chart of a method for manufacturing a metallic-ceramic biomedical implant according to one implementation.

FIG. 5 is an exploded, perspective view of a knee implant comprising two ceramic inserts according to another embodiment.

### DETAILED DESCRIPTION

It will be readily understood that the components of the present disclosure, as generally described and illustrated in the drawings herein, could be arranged and designed in a wide variety of different configurations. Thus, the following more detailed description of the embodiments of the apparatus is not intended to limit the scope of the disclosure, but is merely representative of possible embodiments of the disclosure. In some cases, well-known structures, materials, or operations are not shown or described in detail.

Various embodiments of apparatus, materials, methods, and systems are disclosed herein that relate to biomedical implants, such as modular biomedical implants, and other devices made up of a metallic member, such as a metallic outer member, and an inner ceramic insert, such as a sleeve. The sleeve may be integrally bonded or mechanically coupled within the metallic member so as to prevent, or at least substantially prevent, contact between the metallic member and a second metallic member. The composite metallic/ceramic implant may therefore be designed to mate with a secondary metallic member such that the ceramic serves as an interfacing surface between the two metals and/or metal alloys. This may be useful in order to reduce or prevent pitting, crevice corrosion, fretting, and/or galvanic corrosion, which may occur if the two metallic members were directly joined in contact with one another and/or if they are insufficiently separated from one another.

Some embodiments may be particularly useful in total joint arthroplasty and, more particularly, in total hip arthroplasty. However, other embodiments are contemplated in which the principles disclosed herein may be applied to any other biomedical implant system wherein two similar or dissimilar modular metal components need to be mechanically attached or otherwise coupled to one another. Thus, for example, components formed in accordance with one or more principles, implementations, or embodiments of this disclosure can be shaped and machined into useful human endoprostheses, including but not limited to artificial hips, knees, shoulders, ankles and phalange joints, and/or articulation devices in the spine.

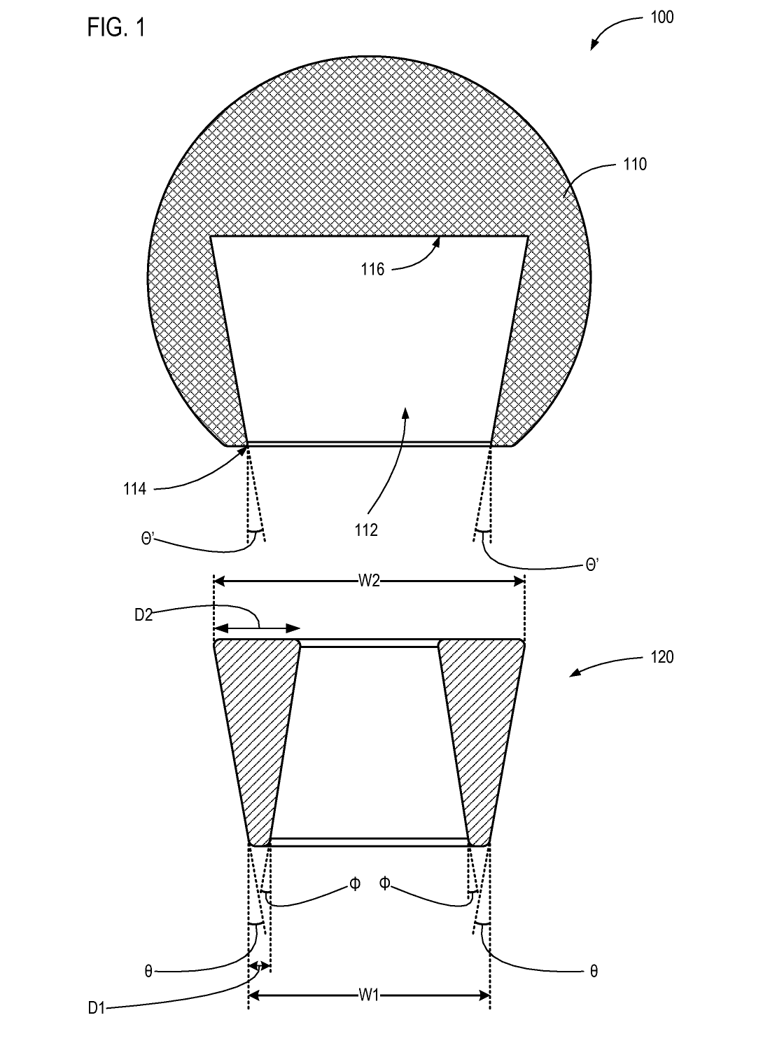

A more particular exemplary embodiment is shown in FIG. 1, which illustrates a femoral head 110 of a modular hip implant 100, along with an insert 120 of hip implant 100. Femoral head 110 may comprise a metal or metal alloy, such as, for example, cobalt-chromium, titanium-aluminum-vanadium, and/or zirconium-niobium alloys. In other embodiments, femoral head 110 may comprise any other metal or metal alloy deemed useful as a component in a total joint arthroplasty or other biomedical implant.

Preferably, the metal or metal alloy of femoral head 110 at least lines a cavity or another portion of the metallic piece that is configured to engage with another metallic piece, or at least a metallic portion of another piece. Thus, femoral head 110 may comprise a cavity 112. Cavity 112 may be lined with one or more of the metals or metal alloys discussed herein. In some embodiments, the entire metallic member/component (i.e., femoral head 110 in its entirety) may comprise the metal or metal alloy material.

Cavity 112 may comprise a reverse taper. Thus, in some embodiments, a peripheral opening 114 of cavity 112 may comprise a first diameter and an internal portion of the cavity, such as terminal internal portion 116 of cavity 112, may comprise a second diameter greater than the first diameter. In the depicted embodiment, the reverse taper of cavity 112 may be gradual from one end of the cavity 112 to the opposite end. However, other embodiments are contemplated in which this need not be the case.

Modular implant 100 may further comprise an insert 120. Insert 120 may comprise a sleeve that may be configured to fit within and be bonded or otherwise coupled to the portion of femoral head 110 that defines cavity 112. Insert 120 may, in certain preferred embodiments, be prepared as a monolithic, sintered ceramic from any number of oxide or non-oxide materials including, without limitation, alumina, zirconia, zirconia-toughened-alumina, silicon nitride, silicon carbide, or mixtures thereof, along with like compositions.

In some embodiments, the ceramic material may comprise a doped silicon nitride (Si₃N₄) having relatively high hardness, tensile strength, elastic modulus, lubricity, and fracture toughness properties. Examples of suitable silicon nitride materials are described, for example, in U.S. Pat. No. 6,881,229 titled “Metal-Ceramic Composite Articulation,” which is incorporated by reference herein in its entirety. Powders of silicon nitride (Si₃N₄) and dopants, such as alumina (Al₂O₃), yttria (Y₂O₃), magnesium oxide, and strontium oxide, can be processed in a conventional manner to form a doped composition of silicon nitride. The dopant amount may be optimized to achieve the highest density and mechanical properties, in some instances. In some embodiments, powders of silicon nitride may be used to form the ceramic implants, either alone or in combination with one or more of the dopants referenced above.

Other examples of suitable silicon nitride materials are described in U.S. Pat. No. 7,666,229 titled “Ceramic-Ceramic Articulation Surface Implants,” which is hereby incorporated by reference in its entirety. Still other examples of suitable silicon nitride materials are described in U.S. Pat. No. 7,695,521 titled “Hip Prosthesis with Monoblock Ceramic Acetabular Cup,” which is also hereby incorporated by reference in its entirety.

In some embodiments, insert 120 may comprise an annular member having a wall defining a wall thickness. In some embodiments, the insert 120 may comprise a wall having a thickness of at least about 1 mm. In some such preferred embodiments, the wall thickness may be between about 1 mm and about 10 mm. Such thicknesses may be needed, or desired, in order to suitably reduce certain problems often associated with modular implants having metallic components that contact or are near each other, such as galvanic corrosion, fretting corrosion, taper corrosion, crevice corrosion, and pitting.

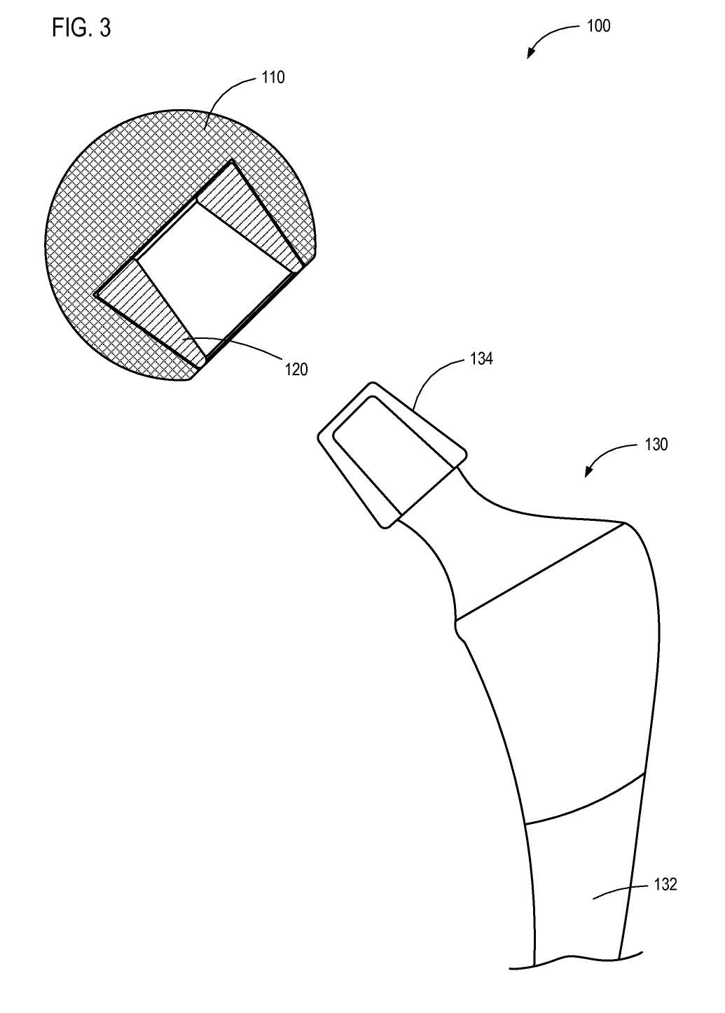

In some embodiments, insert 120 may comprise a cylindrical member comprising a plate portion configured to be positioned adjacent to terminal internal portion 116 of cavity 112. In other words, in such embodiments, insert 120 may comprise a terminal portion comprising a plate that is configured to be positioned adjacent to terminal internal portion 116 of cavity 112 in order to further insulate the metallic material of femoral head 110 from another metallic component, such as a stem of a modular hip implant (see FIG. 3).

Certain prior art hip implants have used hard ceramic coatings, such as titanium nitride (TiN), onto the taper junction of the femoral stem. However, these applied coatings are typically thin films (i.e., <5 μm) and are also susceptible to pitting, cracking, abrasive wear and spallation themselves. They are often therefore not suitable long-term solutions to the general problem of implant corrosion. Providing a thicker barrier between metallic components, as disclosed herein, may ensure separation of the two metallic members and thereby prevent, or at least better inhibit, galvanic corrosion. In some embodiments, the dimensions of the insert may be advantageously designed to also increase the stiffness of the modular joint, thereby further minimizing pitting, crevice, and/or fretting corrosion. For example, as mentioned above, for certain orthopedic implants, the wall thickness of a sleeve embodiment of the insert may be at least 1 mm.

In some embodiments, insert 120 may also comprise a reverse taper. In some embodiments, the reverse taper of insert 120 may be configured to match, or at least substantially match, the reverse taper of cavity 112. For example, in the depicted embodiment, insert 120 may comprise a width W1 at one end and a larger width W2 at an opposite end. In some embodiments, a thickness of a wall defining insert 120 may comprise a thickness D1 at one end and a larger thickness D2 at an opposite end. In some embodiments, the reverse taper may be such that the thickness of the wall and/or width of the insert 120 varies gradually from one end of insert 120 to the opposite end.

In some embodiments, the wall thickness D2 may be between about 1 mm and about 10 mm. Preferably, wall thickness D2 is greater than wall thickness D1. Thus, in some embodiments, an average wall thickness between D1 and D2 may be between about 1 mm and about 10 mm. However, in other embodiments, it may be important that the wall thickness of the thinnest portion of insert 120 (D1) not be less than about 1 mm. Thus, in some such embodiments, D1 may be between about 1 mm and about 10 mm. In such embodiments, D2 may therefore be greater than 1 mm.

In some embodiments, the reverse taper of insert 120 may comprise an angle θ configured such that insert 120 may be incorporated into a metallic femoral head or another component of a biomedical implant through thermal expansion of the head/component upon heating, as described in greater detail below. This angle may be measured with respect to a central axis of insert 120. Similarly, the reverse taper of cavity 112 may comprise an angle θ′. In some embodiments, angle θ may be the same as, or at least substantially the same as, angle θ′.

In some embodiments, insert 120 may further comprise an interior taper, which may define a taper for receipt of another metallic component, such as a neck of an elongated stem of a modular hip implant 100. Thus, in the depicted embodiment, insert 120 comprises an interior surface defining a taper of angle φ. In some embodiments, angle φ may define a 12/14 taper.

Insert 120 may be integrally bonded to cavity 112 of femoral head 110 by any number of methods to ensure a strong cohesive interface. Examples of suitable bonding methods include, for example, press-fitting, shrink-fitting, diffusion bonding, and cementing. In some embodiments comprising reverse tapers, to ensure strong bonding and to prevent separation of the ceramic insert from the metallic member, the selected tolerances of the reverse taper(s) may be designed such that the metallic member (outer member in the case of a femoral head) will expand sufficiently during a thermal heating process to allow the ceramic insert to be placed inside it. Upon cooling, the metallic member may shrink around, or otherwise form a tight bond with, the ceramic insert. The two components may thereby form an excellent mechanical bond that may prevent pull-out or disengagement of the ceramic insert.

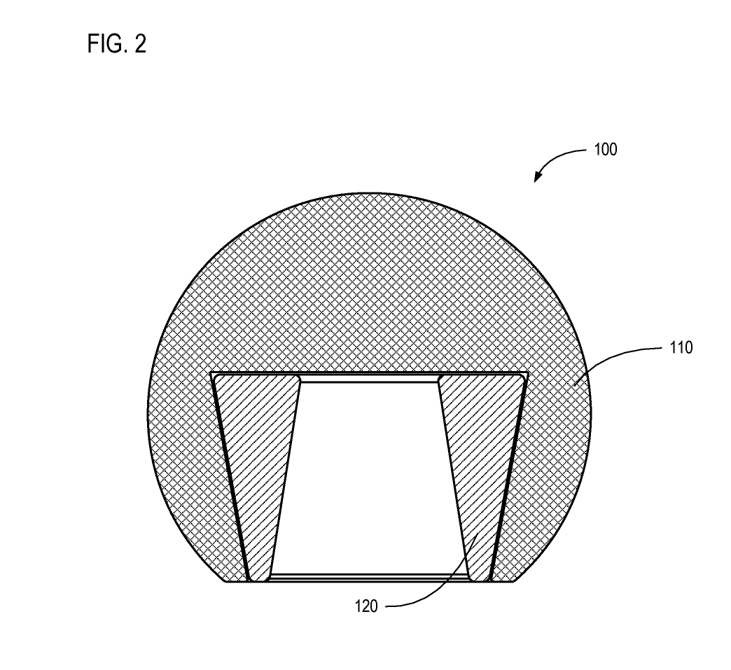

FIG. 2 illustrates the femoral head 110 and insert 120 after insert 120 has been inserted into cavity 112 and bonded with femoral head 110. After this bonding has taken place, the combined metallic femoral head 110 and ceramic insert 120 may be coupled with a secondary metallic component, such as an elongated stem of a modular hip implant. FIG. 3 is an exploded view illustrating the combined femoral head 110 and insert 120 with an elongated femoral component 130 comprising a stem 132 and a neck 134. As illustrated in this figure, the combined metallic-ceramic component made up of femoral head 110 and insert 120 may be coupled with femoral component 130 by inserting neck 134 into the cavity defined by insert 120.

In some embodiments, the inserts disclosed herein may be configured so as to serve as an electric insulator between two metals to eliminate or at least reduce galvanic corrosion. In some embodiments the two metals may be dissimilar metals, since galvanic corrosion typically occurs when there is an electrochemical potential between two adjacent dissimilar metals.

In some embodiments, the methods and/or materials used to form the insert(s) may be designed such that the strength, toughness, and/or modulus of elasticity will be sufficient to minimize micro-motion, thereby reducing or eliminating pitting, crevice and fretting corrosion. Thus, in some embodiments, ceramic materials, such as silicon nitride ceramic materials, may be preferred, since such ceramic materials typically have higher strength, toughness, and/or elasticity values.

Micro-motion between two cylindrical mechanically joined or bonded materials, as disclosed herein, is primarily governed by the elastic modulus of the inner ceramic insert or sleeve multiplied by its second area moment of inertia. Thus, a higher modulus ceramic insert or sleeve coupled with a larger diameter design may markedly reduce micro-motion and minimize pitting, crevice, or fretting corrosion. Because ceramics typically have modulii of elasticity that are substantially higher than metals, providing a ceramic insert, and preferably a monolithic ceramic insert, may be particularly useful in reducing these outcomes. For instance, silicon nitride has an elastic modulus that is substantially higher (about 300 GPa) compared to a CoCr alloy (about 200 GPa) or titanium alloys (about 110 GPa for Ti6Al4V and about 80 GPa for TMZF). Thus, in some embodiments, the modulus of elasticity of the insert may be at least about 80 GPa. In some more preferred such embodiments, the modulus of elasticity of the insert may be at least about 100 GPa.

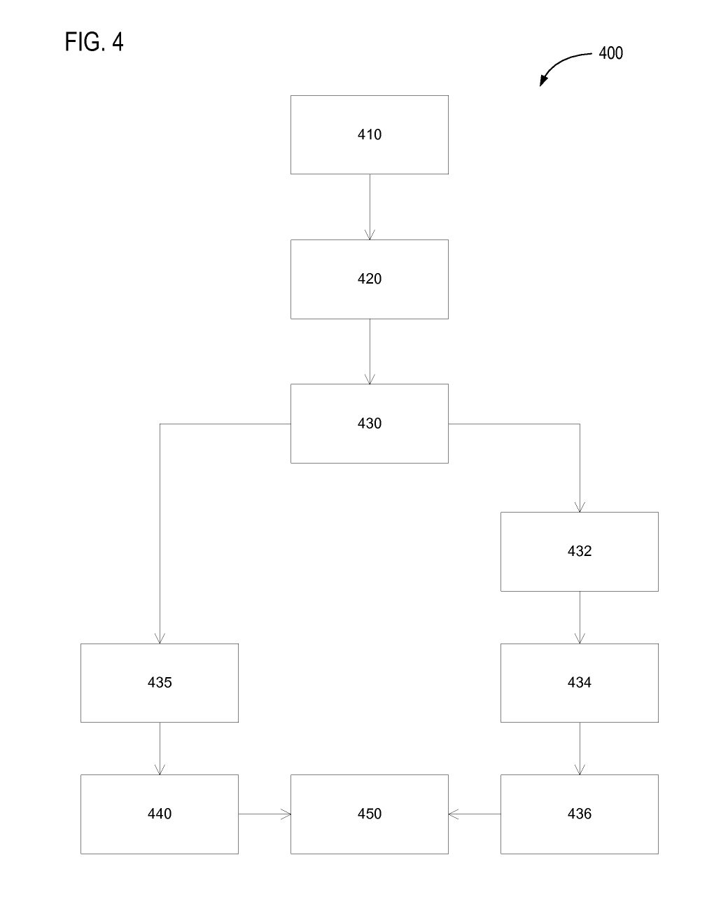

FIG. 4 is a flow chart illustrating an example of a method 400 for manufacturing a metallic-ceramic biomedical implant according to one implementation. Method 400 may begin with step 410, in which a first metallic member comprising at least one of a metal and a metal alloy may be provided. In some implementations, step 410 may therefore comprise manufacturing the first metallic member. Alternatively, step 410 may comprise obtaining the first metallic member from a third party source. In some implementations, the first metallic member may comprise, for example, a femoral head of a hip implant system.

The first metallic member may comprise a cavity comprising a peripheral opening and an internal portion. In some implementations, the cavity may comprise a reverse taper such that the peripheral opening comprises a first diameter and the internal portion comprises a second diameter greater than the first diameter.

At step 420, a second metallic member comprising at least one of a metal and a metal alloy may be provided. The second metallic member may be configured to be coupled with the first metallic member.

At step 430, a ceramic insert comprising a ceramic material may be provided. In some implementations, the ceramic insert may comprise a monolithic ceramic material. The ceramic material may comprise, for example, alumina, zirconia, zirconia-toughened alumina, silicon nitride, and/or silicon carbide.

Following step 430, method 400 may proceed either to step 432 or step 435, either branch of which may result in a suitable coupling of the ceramic insert to the first metallic member. In implementations in which step 430 proceeds to step 432, the ceramic insert may be positioned in the cavity such that the ceramic insert at least partially lines the cavity. In some implementations, the ceramic insert may fully line the cavity so as to prevent contact between the first metallic member and the second metallic member.

In some implementations, the ceramic insert may comprise an external reverse taper such that a first end of the ceramic insert comprises an external diameter of a first length and a second end of the ceramic insert opposite from the first end has an external diameter of a second length, wherein the first length is greater than the second length, and wherein the first end is configured to be received within the cavity adjacent to the internal portion of the cavity.

After positioning the ceramic insert within the cavity, the ceramic insert may be bonded to the first metallic member at step 440. This may be accomplished, for example, by press-fitting, diffusion bonding, or cementing the ceramic insert to the first metallic member within the cavity of the first metallic member.

In implementations in which step 430 proceeds to step 432, the ceramic insert may be shrink-fitted to the first metallic member. Thus, step 432 may comprise heating the first metallic member to expand the dimensions of the cavity and create an expanded cavity within which to fit the ceramic insert. Thus, in some implementations, the ceramic insert may be configured such that it cannot be positioned within the cavity of the first metallic member prior to heating the first metallic member and/or when the first metallic member is at room temperature.

Method 400 may then proceed to step 434, at which point the ceramic insert may be positioned within the heated/expanded cavity. At step 436, the first metallic member may then be allowed to cool with the ceramic insert within the expanded cavity such that the first metallic member shrinks around the ceramic insert and bonds the first metallic member to the ceramic insert.

Step 450 may then comprise coupling the second metallic member to the first metallic member with the ceramic insert at least partially interposed therebetween. In some implementations, the ceramic insert may be fully interposed between the first and second metallic members. In some implementations, step 450 may comprise inserting at least a portion of the second metallic member into a cavity defined at least in part by the ceramic insert within the first metallic member.

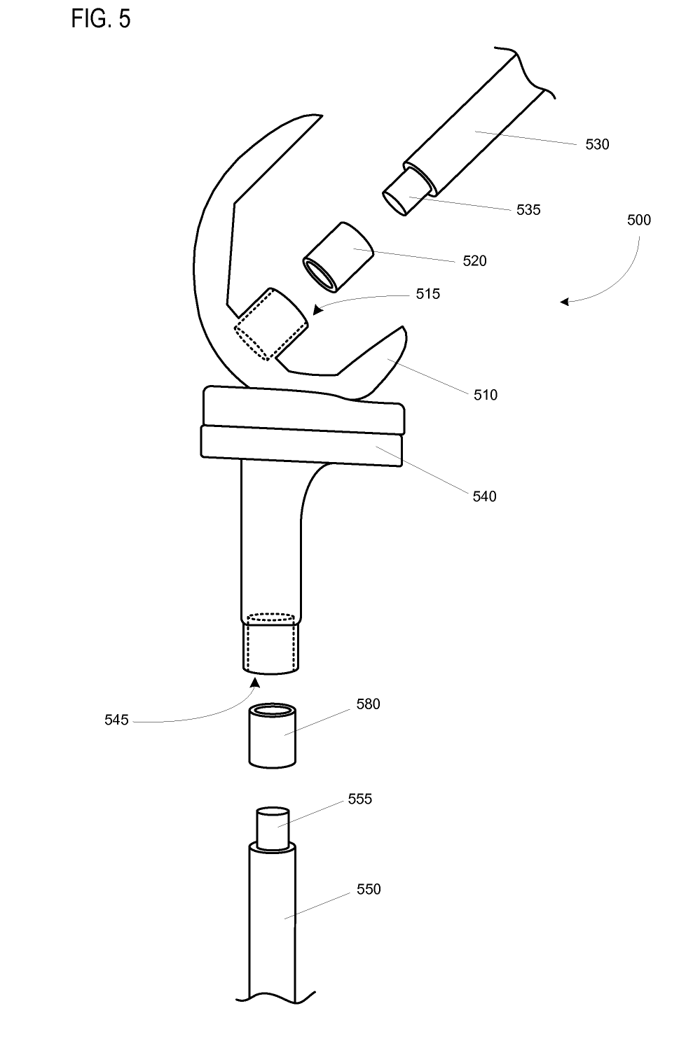

FIG. 5 illustrates another embodiment a knee implant 500 comprising ceramic inserts. Knee implant comprises a femoral component 510, which may comprise an articulating surface and may be configured to replace the natural articulatory surfaces of the human knee joint. Thus, the femoral component 510 may be configured for being coupled to the lower end of a resected femoral bone. More particularly, in the depicted embodiment, a femoral rod 530 may be provided to facilitate direct coupling with the femoral bone. In order to prevent certain undesirable consequences that may be associated with contacting, or close proximity between, adjacent metallic surfaces of rod 530 and a coupling feature, such as opening 515, of femoral component 510, a ceramic insert 520 may be provided.

As previously described, ceramic insert 520 may comprise a sleeve. Sleeve 520 may define an annular shape, as illustrated in FIG. 5. In addition, in preferred embodiments, the ceramic sleeve 520 may comprise a wall having a thickness greater than about 1 mm. In preferred embodiments, the ceramic sleeve 520 further comprises a monolithic ceramic insert, so as to enhance the ability of insert 520 to prevent or at least inhibit pitting, crevice, fretting, and/or galvanic/taper corrosion.

In addition, as also previously described, insert 520 may comprise a taper on its inner and/or outer surfaces. However, in the depicted embodiment, no such tapers are present. Thus, insert 520 may be coupled with coupling feature/opening 515 of femoral component 510 by, for example, bonding methods, such as press-fitting, diffusion bonding, and cementing. In embodiments comprising an exterior reverse taper, a shrink-fit process, as described above, may be used to couple insert 520 with femoral component 510.

In some embodiments, a tibial component 540 may also be provided. Tibial component 540 may comprise, for example, a monoblock structure configured for being secured to the upper end of a resected tibial bone. As those of ordinary skill in the art will appreciate, tibial component 540 may be configured to define one or more contoured surfaces, such as a spaced pair of concave bearing surfaces (not visible in FIG. 5), which may be configured to receive and allow for articulation of condyles positioned on femoral component 510.

A tibial rod 550 or another similar component, such as a metaphyseal cone, may be configured to directly couple with a resected portion of a patient's tibia at one end, and may be configured to couple with tibial component 540 at the opposite end. In some embodiments, a second ceramic insert 580 may be provided to be positioned in between a coupling feature, such as opening 545 of tibial component 540, and tibial rod 550.

In some embodiments a portion of the tibial rod 550 and/or femoral rod 530 may be configured with tips 555 and 535, respectively. Such tips may be configured to receive sleeves 520 and 580, respectively, to a preconfigured depth. This may be accomplished by providing tips 535 and 555 that have a reduced diameter, and thereby provide a shelf or stop, as illustrated in FIG. 5, for sleeves 520/580 and/or a peripheral portion of openings 515/545.

It will be understood by those having skill in the art that changes may be made to the details of the above-described embodiments without departing from the underlying principles presented herein. For example, any suitable combination of various embodiments, or the features thereof, is contemplated.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Throughout this specification, any reference to “one embodiment,” “an embodiment,” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment. Unless otherwise noted, the terms “a” or “an” are to be construed as meaning “at least one of.” In addition, for ease of use, the words “including” and “having” are interchangeable with and have the same meaning as the word “comprising.” Recitation of the term “first” with respect to a feature or element does not necessarily imply the existence of a second or additional such feature or element.

Similarly, it should be appreciated that in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, inventive aspects lie in a combination of fewer than all features of any single foregoing disclosed embodiment.

It will be apparent to those having skill in the art that changes may be made to the details of the above-described embodiments without departing from the underlying principles set forth herein. The scope of the present invention should, therefore, be determined only by the following claims.

1. A modular biomedical implant, comprising:
a first metallic member comprising at least one of a metal and a metal alloy;
a second metallic member comprising at least one of a metal and a metal alloy; and
a monolithic ceramic insert comprising a ceramic material positioned in between the first metallic member and the second metallic member so as to at least substantially prevent contact between the first metallic member and the second metallic member.

2. The modular biomedical implant of claim 1, wherein the ceramic material comprises at least one of alumina, zirconia, zirconia-toughened alumina, silicon nitride, and silicon carbide.

3. The modular biomedical implant of claim 2, wherein the ceramic material comprises a silicon nitride ceramic.

4. The modular biomedical implant of claim 1, wherein the first metallic member comprises a femoral head of a modular hip implant, and wherein the second metallic member comprises a neck of the modular hip implant configured to be coupled with the femoral head.

5. The modular biomedical implant of claim 4, wherein the monolithic ceramic insert comprises a sleeve positioned within a cavity formed within the femoral head.

6. The modular biomedical implant of claim 5, wherein the cavity comprises a reverse taper such that a peripheral opening of the cavity comprises a first diameter and an internal portion of the cavity comprises a second diameter, wherein the first diameter is less than the second diameter.

7. The modular biomedical implant of claim 6, wherein the sleeve comprises an external reverse taper such that a first end of the sleeve has an external diameter of a first length and a second end of the sleeve opposite from the first end has an external diameter of a second length, wherein the first length is greater than the second length, and wherein the first end is configured to be received within the cavity adjacent to the internal portion of the cavity.

8. The modular biomedical implant of claim 1, wherein the monolithic ceramic insert is positioned and configured to wholly prevent any contact between the first metallic member and the second metallic member.

9. The modular biomedical implant of claim 1, wherein the monolithic ceramic insert comprises a wall having a thickness of at least about 1 mm.

10. The modular biomedical implant of claim 1, wherein the first metallic member comprises an articulating femoral component of a knee implant, and wherein the second metallic member comprises an intramedullary rod of the knee implant configured to be coupled with the femoral component.

11. The modular biomedical implant of claim 1, further comprising:
a third metallic member comprising at least one of a metal and a metal alloy;
a fourth metallic member comprising at least one of a metal and a metal alloy; and
a second monolithic ceramic insert comprising a ceramic material positioned in between the third metallic member and the fourth metallic member so as to at least substantially prevent contact between the third metallic member and the fourth metallic member.

12. The modular biomedical implant of claim 11, wherein the first metallic member comprises an articulating femoral component of a knee implant, wherein the second metallic member comprises an intramedullary rod of the knee implant configured to be coupled with the femoral component, wherein the third metallic member comprises a tibial component of the knee implant, wherein the fourth metallic member comprises at least one of a rod and a metaphyseal cone of the knee implant.

13. A modular hip implant, comprising:
a femoral head comprising at least one of a metal and a metal alloy, wherein the femoral head comprises a cavity, wherein the cavity comprises a peripheral opening having a first diameter, and wherein the cavity comprises a terminal portion having a second diameter greater than the first diameter; and
a ceramic insert comprising a ceramic material positioned within the cavity so as to at least substantially prevent contact between a portion of the femoral head defining the cavity and a second metallic member of the modular hip implant, wherein the ceramic insert comprises an exterior surface configured to engage the portion of the femoral head defining the cavity and an interior surface configured to engage the second metallic member, wherein the exterior surface extends between a first end and a second end, and wherein a diameter of the exterior surface at the first end is greater than a diameter of the exterior surface at the second end.

14. The modular hip implant of claim 13, wherein the cavity defines a reverse taper extending from the peripheral opening to the terminal portion.

15. The modular hip implant of claim 14, wherein the exterior surface of the ceramic insert defines a reverse taper.

16. The modular hip implant of claim 15, wherein the reverse taper of the cavity extends at a first angle relative to a central axis of the cavity, wherein the reverse taper of the ceramic insert extends at a second angle relative to a central axis of the ceramic insert, and wherein the first angle is at least substantially identical to the second angle.

17. The modular hip implant of claim 13, further comprising a hip stem comprising a neck, wherein the hip stem is configured to be coupled with an upper end of a patient's femur, and wherein the neck is configured to be received in the ceramic insert.

18. The modular hip implant of claim 13, wherein the ceramic insert comprises a sleeve, and wherein the sleeve defines an annular shape.

19. The modular hip implant of claim 18, wherein the sleeve comprises a wall having a thickness greater than about 1 mm.

20. The modular hip implant of claim 13, wherein the ceramic insert comprises a monolithic ceramic insert.

21. A method for manufacturing a biomedical implant, the method comprising the steps of:
providing a first metallic member comprising at least one of a metal and a metal alloy, wherein the first metallic member comprises a cavity comprising a peripheral opening and an internal portion, and wherein the cavity comprises a reverse taper such that the peripheral opening comprises a first diameter and the internal portion comprises a second diameter greater than the first diameter;
providing a second metallic member comprising at least one of a metal and a metal alloy configured to be coupled with the first metallic member;
providing a ceramic insert comprising a ceramic material, wherein the ceramic insert comprises an external reverse taper such that a first end of the ceramic insert comprises an external diameter of a first length and a second end of the ceramic insert opposite from the first end has an external diameter of a second length, wherein the first length is greater than the second length, and wherein the first end is configured to be received within the cavity adjacent to the internal portion of the cavity;
positioning the ceramic insert in the cavity such that the ceramic insert at least partially lines the cavity;
bonding the ceramic insert to the first metallic member; and
inserting at least a portion of the second metallic member into the cavity defined at least in part by the ceramic insert.

22. The method of claim 21, wherein the ceramic insert is positioned and bonded to the first metallic member so as to wholly line the cavity of the first metallic member such that contact is prevented between the first metallic member and the second metallic member.

23. The method of claim 21, wherein the step of bonding the ceramic insert to the first metallic member comprises shrink-fitting the ceramic insert to the first metallic member.

24. The method of claim 23, wherein the step of shrink-fitting the ceramic insert to the first metallic member comprises:
heating the first metallic member to expand the dimensions of the cavity and create an expanded cavity;
positioning the ceramic insert within the expanded cavity; and
allowing the first metallic member to cool with the ceramic insert within the expanded cavity such that the first metallic member shrinks around the ceramic insert.
